(19) United States
(12) Patent Application Publication
(10) Pub. No.: US 2011/0202108 A1
(43) Pub. Date: Aug. 18, 2011

(54) ELECTRICAL MENORRHAGIA TREATMENT

(75) Inventor: Yossi Gross, Moshav Mazor (IL)

(73) Assignee: RAINBOW MEDICAL LTD., Herzliya (IL)

(21) Appl. No.: 12/707,897

(22) Filed: Feb. 18, 2010

(51) Int. Cl. A61N 1/04

(52) U.S. Cl. 607/46

(57) ABSTRACT

Apparatus and methods are provided for treating menorrhagia of a subject. At least one electrode is coupled to a pelvic site of the subject. A control unit reduces nitric oxide production by pelvic tissue of the subject by driving the electrode to drive an electric current into the pelvic site of the subject. Other embodiments are also described.

### CROSS-REFERENCES TO RELATED APPLICATIONS

The present application is related to PCT Application PCT/IL2008/001686 (published as WO 09/081,411) to Gross, filed Dec. 28, 2008, which is incorporated herein by reference.

### FIELD OF APPLICATIONS OF THE INVENTION

Some applications of the present invention relate to medical apparatus. Specifically, some applications of the present invention relate to an electrode device for treating menorrhagia.

### BACKGROUND

Menorrhagia is abnormally heavy and prolonged menstrual bleeding at regular intervals. Menorrhagia may be due to abnormal blood clotting or disorders of the endometrial lining of the uterus. Depending on the cause of menorrhagia, it may be associated with abnormally painful periods (dysmenorrhea).

PCT Publication WO 09/081,411 to Gross describes a manual insertion device configured to be inserted by the subject into a vagina of the subject and removed by the subject from the vagina of the subject. At least one electrode is coupled to the insertion device. A control unit is configured to chronically increase nitric oxide production by tissue of the subject by driving the electrode to drive an electric current into the vagina.

An article entitled, “Electrical stimulation has no adverse effect on pregnant rats and fetuses,” by Yongjin Wang et al., in The Journal of Urology, Vol. 162, pp. 1785-1787, November 1999 describes how electrical stimulation has been considered a contraindication in pregnant women with various voiding dysfunctions, because of the potential to cause teratogenicity or abortion. In their article, they state that it is not known whether electrical stimulation can cause fetal malformation or abortion. The purpose of this study was to evaluate whether electrical stimulation has any adverse effect on pregnant rats and fetuses. Electrical stimulation was not shown to have any adverse effect on pregnant rats and their fetuses. The authors suggest that termination of pregnancy is not advised for prospective mothers when electrical stimulation has been performed inadvertently in early pregnancy.

An article entitled, “Nitric oxide in the endometrium,” by Cameron I T and Campbell S, in Human Reproduction Update, Vol. 4, No. 5, pp. 565-569, 1998 describes nitric oxide (NO) as an important mediator of paracrine interactions, especially within the vascular system. It is a powerful inhibitor of platelet aggregation and a potent vasodilator. NO is also a neurotransmitter and it plays a role in cell-mediated cytotoxicity. NO-generating enzymes (nitric oxide synthases, NOS) have been described in the endometrium of a number of species, suggesting that NO might be involved in endometrial function. In human endometrium, endothelial NOS and inducible NOS have been localized to glandular epithelium in the non-pregnant uterus. Weak inducible NOS immunoreactivity has been observed in decidualized stromal cells. NO might participate in the initiation and control of menstrual bleeding. Furthermore, it may play a part in the inhibition of platelet aggregation within the endometrium, where menstrual hemostasis is thought to occur primarily by vasoconstriction rather than clot organization. Endometrially derived NO could also suppress myometrial contractility. Recent attention has focused on the part that NO might play in maintaining myometrial quiescence during pregnancy. NO also appears to relax the non-pregnant myometrium, an action which could be exploited for the medical treatment of primary dysmenorrhea.

U.S. Pat. No. 5,188,122 to Phipps et al. describes apparatus for applying electromagnetic radiation to the pelvic cavity. The apparatus comprises a first electrode which is inserted into the pelvic cavity in use. A second electrode is also provided, and an electromagnetic generating circuit is coupled to each electrode. The frequency generated by the generating circuit in use is described as being emitted by one of the electrodes and received at the other electrode so that the temperature of the cells adjacent to the first electrode is increased above the normal cell temperature. The apparatus is described as being particularly useful for exposing the endometrium to histotoxic temperature and to thus achieve endometrial destruction. Typically, the electromagnetic radiation is radio frequency radiation which is preferably in the range of 500 kHz to 500 MHz. The end of the first electrode may be curved in order to allow access to the cornua regions of the uterine cavity and to improve endometrial contact.

U.S. Pat. No. 5,948,762 to Garfield et al. describes treatment for dysmenorrhea, dysfunctional uterine bleeding, preterm labor and postpartum labor in female mammals. They describe inhibiting uterine contractility by administering thereto a nitric oxide synthase substrate, a nitric oxide donor or both, optionally in combination with one or more of a prostaglandin inhibitor, a prostacyclin-mimetic, a progestin, an oxytocin antagonist or a beta-agonist in an amount effective to ameliorate the symptoms thereof. Inadequate menses is treated and induction of abortion or stimulation of labor in a pregnant female is achieved by uterine contractility stimulation by administering thereto a nitric oxide inhibitor, either alone or optionally in a combination of progesterone antagonist, an oxytocin or -oxytocin analogue antagonist or a prostaglandin.

U.S. Pat. No. 5,916,173 to Kirsner describes a method for monitoring fertility status in a female mammal comprising the steps of placing in the vagina of the female a probe having opposed electrodes, orienting the probe so that at least one of the electrodes is touching the cervix of the female, measuring across the electrodes at least one physical parameter indicative of the phase of the female fertility cycle, and comparing the value of the parameter with a reference. A probe useful in practicing this method comprises an elongated body having an insertion end, two electrodes attached to said body at the insertion end, and orienting means for orienting the body so that at least one of the electrodes touches the cervix of the female.

Pfizer (New York City, N.Y.) manufactures Cleocin®, a clindamycin phosphate vaginal cream described as an intravaginal treatment for vaginal odor caused by bacterial vaginosis.

Galderma (France, Canada, Brazil) manufactures metronidazole marketed under the trade name Metrogel®, which is described as a vaginal gel used for the treatment of bacterial vaginosis.
U.S. Pat. No. 7,229,403 to Schock et al.
U.S. Pat. No. 7,206,637 to Salo
U.S. Pat. No. 6,939,345 to KenKnight et al.
U.S. Pat. No. 6,896,651 to Gross et al.
U.S. Pat. No. 6,871,092 to Piccone
U.S. Pat. No. 6,865,416 to Dev et al.
U.S. Pat. No. 6,862,480 to Cohen et al.
U.S. Pat. No. 6,845,267 to Harrison et al.
U.S. Pat. No. 6,824,561 to Soykan et al.
U.S. Pat. No. 6,810,286 to Donovan et al.
U.S. Pat. No. 6,741,895 to Gafni et al.
U.S. Pat. No. 6,562,297 to Bonstein
U.S. Pat. No. 6,485,524 to Strecker
U.S. Pat. No. 6,479,045 to Bologna et al.
U.S. Pat. No. 6,463,323 to Conrad-Vlasak et al.
U.S. Pat. No. 6,432,037 to Eini et al.
U.S. Pat. No. 6,348,640 to Navot
U.S. Pat. No. 6,347,247 to Dev et al.
U.S. Pat. No. 6,245,103 to Stinson
U.S. Pat. No. 6,200,259 to March
U.S. Pat. No. 6,139,538 to Houghton et al.
U.S. Pat. No. 6,086,527 to Talpade
U.S. Pat. No. 6,063,042 to Navot
U.S. Pat. No. 6,058,331 to King
U.S. Pat. No. 6,030,375 to Anderson et al.
U.S. Pat. No. 5,906,641 to Thompson et al.
U.S. Pat. No. 5,830,848 to Harrison et al.
U.S. Pat. No. 5,800,502 to Eoutos
U.S. Pat. No. 5,800,501 to Sherlock et al.
U.S. Pat. No. 5,562,717 to Tippey
U.S. Pat. No. 5,361,627 to Levesque
U.S. Pat. No. 5,324,323 to Bui
U.S. Pat. No. 5,046,511 to Mauer et al.
U.S. Pat. No. 4,827,946 to Kaali et al.
U.S. Pat. No. 3,794,024 to Kokx
U.S. Design Pat. 415835 to Malewicz et al.
US Patent Application Publication 1998/34677 to Hilburg et al.
US Patent Application Publication 2002/0103454 to Sackner et al.
US Patent Application Publication 2003/0036773 to Whitehurst et al.
US Patent Application Publication 2003/0204206 to Padua et al.
US Patent Application Publication 2003/057264 to Geiser et al.
US Patent Application Publication 2004/0039417 to Soykan et al.
US Patent Application Publication 2006/0276844 to Alon et al.
US Patent Application Publication 2007/0196428 to Glauser et al.
US Patent Application Publication 2007/0248676 to Stamler et al.
PCT Publication WO 00/002501 to Benjamin et al.
PCT Publication WO 04/014456 to Allen et al.
PCT Publication WO 05/092439 to Fox et al.
PCT Publication WO 06/064503 to Belsky et al.
PCT Publication WO 06/094273 to White et al.
PCT Publication WO 06/123346 to Alon et al.
PCT Publication WO 07/059,990 to Boyd et al.
PCT Publication WO 07/106,533 to Stern et al.
PCT Publication WO 07/113,833 to Cahan et al.
European Patent Application Publication EP 0 109 935 A1 to Charmillot et al.
Capdevila, “Dysfunctional uterine bleeding and dysmenorrhea,” European Journal of Contraception Reproduction Health Care 2(4):229-37 (1997)
Chwalisz K et al., “Role of nitric oxide in implantation and menstruation,” Hum Reprod (3):96-11 (2000)
Gragasin F S et al., “The neurovascular mechanism of clitoral erection: nitric oxide and cGMP-stimulated activation of BKCa channels,” The FASEB Journal 18:1382-1391, 2004
Gorodeski G I, “NO increases permeability of cultured human cervical epithelia by cGMP-mediated increase in G-actin,” Am J Physiol Cell Physiol 278:942-952 2000
Morlin et al., “Nitric oxide increases endocervical secretion at the ovulatory phase in the female,” Acta Obstet Gynecol Scand 84:883-886 2005
Morlin et al., “Nitric oxide induces endometrial secretion at implantation time,” Acta Obstet Gynecol Scand 84:1029-1034 2005
Munarriz K R et al., “A review of the physiology and pharmacology of peripheral (vaginal and clitoral) female genital arousal in the animal model,” J Urol. 170(2 Pt 2):S40-4, 2003
Norman et al., “Nitric oxide in the human uterus,” Reviews of Reproduction 1:61-68 1996
Pacher et al. “Topical administration of novel nitric oxide donor, linear polyethylenimine-nitric oxide/nucleophile adduct (DS1), selectively increases vaginal blood flow in anesthetized rats,” International Journal of Impotence Research 15:461-464, 2003
Sobel et al., “Efficacy of clindamycin vaginal ovule (3-day treatment) vs. clindamycin vaginal cream (7-day treatment) in bacterial vaginosis,” Infect Dis Obstet Gynecol 9:9-15, 2001
Vachon P S et al., “Increases in clitoral and vaginal blood flow following clitoral and pelvic plexus nerve stimulations in the female rat,” Int J Impot Res. 12(1):53-7, 2000

### SUMMARY

For some applications of the present invention, a subject is identified as suffering from menorrhagia. An insertion device is manually inserted into the subject's vagina to reduce nitric oxide production by pelvic tissue of the subject. The device is typically inserted by the subject in accordance with a schedule related to the timing of the subject's menstrual period and/or in response to an onset of heavy bleeding or menstrual pain.

Typically, the apparatus is tampon-shaped and comprises a control unit and one or more electrodes. The control unit is configured to drive the electrodes to drive an electrical current into the subject's vagina in order to reduce nitric oxide production by pelvic tissue of the subject.

For some applications, the insertion device is manually inserted into the subject's vagina to enhance nitric oxide production by pelvic tissue of the subject. In one such application, the control unit drives a current into the subject's vagina, generating a release of nitric oxide from the cervix, and/or tissue in the vicinity of the cervix of the subject. This release of nitric oxide causes the cervix to relax slightly, facilitating the release of clots from the uterus, thereby reducing the number of days of bleeding in a period of a subject and/or reducing the pain associated with the period.

In another application, the insertion device is inserted by the subject when the subject is ovulating, or in a window of 2-3 days prior to or following ovulation. The control unit drives a current into the subject's vagina in order to increase nitric oxide production by pelvic tissue, thereby increasing the fertility of the subject.

In an alternative application, the insertion device is automatically or manually activated, or inserted by the subject, in response to a decline in fetal heartbeat. The control unit drives a current into the subject's vagina in order to increase nitric oxide production, thereby increasing blood supply to the fetus or fetuses. In yet another application, the insertion device is inserted by the subject in response to a multiple gestation pregnancy in order to increase blood supply to the fetuses, even in the absence of detection of reduced fetal heartbeat. For some applications, the insertion device is inserted by the subject in order to increase blood supply to a single fetus, even in the absence of detection of reduced fetal heartbeat.

For some applications, one or more electrode units are surgically implanted onto a nerve innervating the uterus of the subject, for example, the inferior hypogastric plexus, the uterovaginal plexus, and/or the pelvic splanchnic nerve. In such applications, the control unit is configured to drive the electrode units to drive a current into the nerve, to reduce nitric oxide production. Alternatively or additionally, the control unit is configured to drive a pain-reduction current into the nerve of the subject to reduce pain associated with menorrhagia.

Although some treatments are described herein as being applied to a subject via electrodes of an insertion device, the scope of the invention includes applying such treatments to the subject via electrodes that are coupled to a nerve. Similarly, although other treatments are described herein as being applied to a subject via electrodes that are coupled to a nerve, the scope of the invention includes applying such treatments to the subject via electrodes of an insertion device.

There is therefore provided, in accordance with some applications of the present invention, apparatus for treating menorrhagia of a subject, including:

at least one electrode, configured to be coupled to a pelvic site of the subject; and

a control unit, configured to reduce nitric oxide production by pelvic tissue of the subject by driving the electrode to drive an electric current into the pelvic site of the subject.

For some applications, the apparatus further includes a sensor configured to sense a level of bleeding of the subject and to generate a signal in response thereto, and the control unit is configured to receive the signal and to reduce the nitric oxide production in response to the signal.

For some applications, the control unit is configured to configure the electric current to have a frequency that is between 50 Hz and 150 Hz.

For some applications, the at least one electrode is configured to be inserted by the subject into a vagina of the subject and removed by the subject from the vagina of the subject.

For some applications, the at least one electrode is configured to be inserted into the vagina of the subject in accordance with a schedule related to timing of a menstrual period of the subject.

For some applications, a total length of the apparatus is 4-10 cm.

For some applications, the control unit is configured to configure the electric current to have an amplitude that is between 1 mA and 15 mA.

For some applications, the control unit is configured to configure the electric current to have an amplitude that is between 5 mA and 15 mA.

For some applications, the at least one electrode is configured to be coupled to a nerve that innervates a uterus of the subject.

For some applications, the at least one electrode is configured to be coupled to a nerve of the subject selected from the group consisting of an inferior hypogastric plexus, a uterovaginal plexus, and a pelvic splanchnic nerve.

For some applications, the control unit is configured to drive a pain-reduction current into the nerve of the subject to reduce pain associated with menorrhagia.

For some applications, the control unit is configured to drive into the nerve of the subject a collision blocking current configured to reduce pain associated with the menorrhagia.

For some applications, the control unit is configured to reduce nitric oxide production by using a first signal protocol, and to reduce pain by driving the pain reduction current into the subject's nerve using a second signal protocol, different from the first signal protocol.

There is further provided, in accordance with some applications of the present invention, a method, including:

identifying a subject as suffering from menorrhagia; and

in response to the identifying:
driving an electric current into a pelvic site of the subject; and
configuring the electric current to reduce nitric oxide production by pelvic tissue of the subject.

For some applications, configuring the electric current includes sensing a level of pain of the subject and configuring the electric current in response to the sensed level of pain.

For some applications, the method further includes implanting the electrode in the pelvic site of the subject, and driving the electric current includes driving the electric current via the electrode while the electrode is implanted in the pelvic site.

There is additionally provided, in accordance with some applications of the present invention, apparatus for treating menstrual pain of a subject, including:

at least one menstrual cramp sensor, configured to be coupled to a pelvic site of the subject and to generate a signal indicative of menstrual pain; and

a control unit, configured to receive the signal and to relieve the menstrual pain by driving an electric current into tissue of the subject.

For some applications, the apparatus includes a manual insertion device, which includes the sensor and the control unit.

For some applications, the apparatus is configured to be surgically implanted in the subject.

For some applications, the sensor includes an electrode.

For some applications, the control unit is configured to receive an electromyographic signal from the electrode, which varies in response to menstrual cramps of the subject.

For some applications, the control unit is configured to drive the electric current via the electrode.

For some applications, the control unit is configured to reduce nitric oxide production by pelvic tissue of the subject, by driving the electrode to drive the electric current.

There is further provided, in accordance with some applications of the present invention, apparatus for increasing fertility of a subject, including:

at least one electrode, configured to be coupled to a pelvic site of the subject; and

a control unit, configured to increase fertility of the subject by increasing nitric oxide production by tissue of the subject by driving an electric current into the tissue.

For some applications, the apparatus further includes an ovulation sensor, configured to sense ovulation of the subject and to generate a signal in response thereto, and the control unit is configured to receive the signal and to increase the nitric oxide production in response to receiving the signal.

For some applications, the at least one electrode is configured to be inserted by the subject into a vagina of the subject and removed by the subject from the vagina of the subject.

For some applications, the control unit is configured to configure the current to have a frequency that is between 3 Hz and 50 Hz.

For some applications, the control unit is configured to configure the current to have a frequency that is between 10 Hz and 20 Hz.

For some applications, the control unit is configured to configure the current to have an amplitude that is between 1 mA and 7 mA.

For some applications, the control unit is configured to configure the current to have an amplitude that is between 1 mA and 3 mA.

There is further provided, in accordance with some applications of the present invention, a method for increasing fertility of a subject, including:

identifying a subject who wants to become pregnant; and

in response to the identifying, driving an electric current into a pelvic site of the subject configured to increase fertility of the subject by enhancing nitric oxide production by pelvic tissue of the subject.

For some applications, the pelvic site includes a vagina of the subject, the method further includes inserting an electrode into the vagina, and driving the electric current includes driving the electric current via the electrode while the electrode is inserted.

For some applications, inserting the electrode includes inserting the electrode into the vagina of the subject in accordance with a schedule related to timing of ovulation of the subject.

There is additionally provided, in accordance with some applications of the present invention, apparatus for increasing blood supply to a fetus within a subject, including:

at least one electrode configured to be coupled to a pelvic site of the subject; and

a control unit, configured to enhance fetal blood supply by increasing nitric oxide production of tissue of the subject, by driving the electrode to drive an electric current into the pelvic site of the subject.

For some applications, the apparatus further includes a sensor configured to sense a heartbeat of the fetus and to generate a signal indicative of the fetal heartbeat, and the control unit is configured to enhance the fetal blood supply in response to identifying a decline of the fetal heartbeat.

For some applications, the apparatus is configured to be surgically implanted in the subject.

For some applications, the at least one electrode is configured to be inserted by the subject into a vagina of the subject and removed by the subject from the vagina of the subject.

For some applications, the control unit is configured to configure the current to have a frequency that is between 3 Hz and 50 Hz.

For some applications, the control unit is configured to configure the current to have a frequency that is between 10 Hz and 20 Hz.

For some applications, the control unit is configured to configure the current to have an amplitude that is between 1 mA and 7 mA.

For some applications, the control unit is configured to configure the current to have an amplitude that is between 1 mA and 3 mA.

There is additionally provided, in accordance with some applications of the present invention, a method, including:

identifying a subject who is pregnant with a fetus; and

in response to the identifying, driving an electric current into a pelvic site of the subject configured to increase blood supply to the fetus by enhancing nitric oxide production by pelvic tissue of the subject.

For some applications, driving the current includes driving the current even in the absence of sensing a decline in fetal heartbeat within an immediately preceding 30 minute period.

For some applications, identifying includes identifying that the subject is pregnant with more than one fetus.

The present invention will be more fully understood from the following detailed description of embodiments thereof, taken together with the drawings, in which:

### BRIEF DESCRIPTION OF THE DRAWINGS

FIG. 1 is a schematic illustration of an insertion device, in accordance with some applications of the present invention; and

FIG. 2 is a schematic illustration of a nerve cuff, in accordance with some applications of the present invention.

### DETAILED DESCRIPTION OF EMBODIMENTS

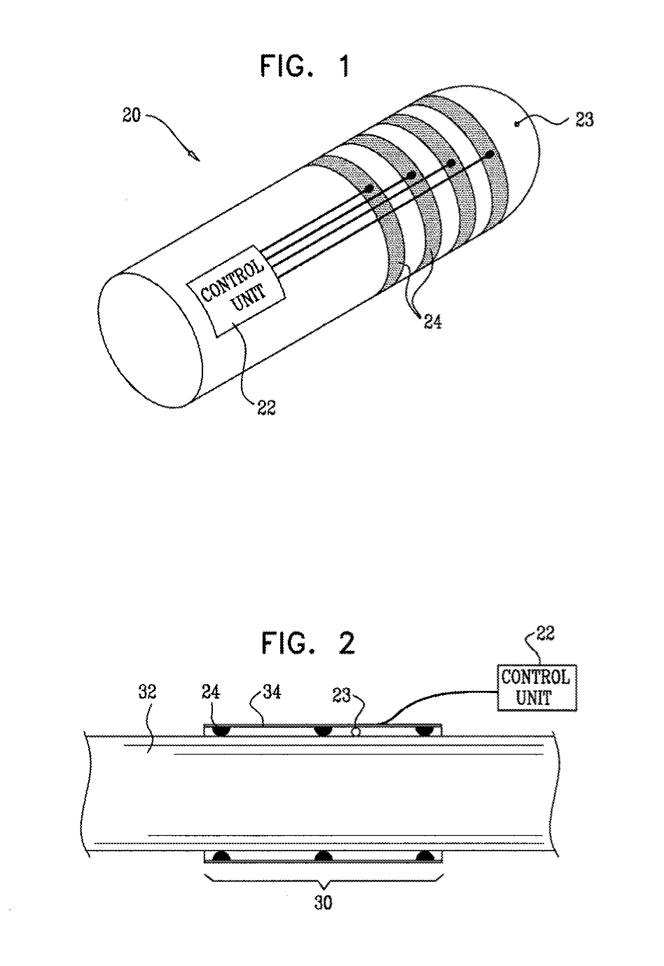

Reference is now made to FIG. 1, which is a schematic illustration of a manual insertion device 20 configured to be inserted into a subject's vagina, in accordance with some applications of the present invention. Manual insertion device 20 comprises a control unit 22, which is coupled to one or more electrodes 24 via electrical leads, and is configured to drive the electrodes to drive an electrical current into a pelvic site of the subject, e.g., the subject's vagina. For some applications, insertion device 20 is configured to reduce nitric oxide production by pelvic tissue of the subject, by driving the current into the vagina. Typically, manual insertion device 20 is tampon-shaped and, for example, about 4-10 cm in length.

For some applications, a subject is identified as suffering from menorrhagia, and device 20 is inserted as a treatment for the menorrhagia. Insertion device 20 is typically inserted by the subject in response to menstrual pain or in accordance with a schedule related to the timing of the subject's menstrual period.

For some applications, device 20 comprises a sensor 23 which is configured to sense the onset of heavy bleeding, and, in response thereto, control unit 22 initiates the driving of the current. As appropriate, the sensor may comprise a pH sensor, an electrical sensor, an electromyographic sensor that senses muscle activity associated with menstrual cramps, or a mechanical sensor, each of which is configured to alert the control unit of the initiation of heavy bleeding. Alternatively, control unit 22 initiates the driving of the current in response to detection that it is disposed within the subject's vagina. For example, electrodes 24 may be coated with a dissolvable coating that dissolves when brought into contact with fluid of the vagina (e.g., vaginal discharge, or blood), and the dissolving of the coating allows the current to flow.

Typically, control unit 22 reduces nitric oxide production by the subject's pelvic tissue by driving electrodes 24 to drive an electric current into the pelvic site of the subject having a frequency of between 50 Hz and 150 Hz, for example, between 50 Hz and 100 Hz. Further typically, control unit 22 drives each of the electrodes 24 to drive a current into the pelvic site of the subject having an amplitude of between 1 mA and 15 mA, e.g., between 5 mA and 15 mA.

For some applications, insertion device 20 increases nitric oxide production by pelvic tissue of the subject, by driving a current into the vagina. In one such application, insertion device 20 is inserted by the subject in accordance with a schedule related to the timing of the menstrual period of the subject, in order to reduce the number of days of the subject's menstrual period. Typically, electrodes 24 are coupled to the tampon-shaped device 20 at its distal tip.

Control unit 22 is configured to drive electrodes 24 to drive an electrical current into the subject's vagina, to increase the nitric oxide production by the cervix and/or tissue in the vicinity of the cervix. This release of nitric oxide in the vicinity of the cervix causes the cervix to relax slightly, facilitating the release of clots from the uterus of the subject, and thereby reducing the number of days and/or severity of the subject's menstrual period. Typically, control unit 22 increases nitric oxide production by the subject's pelvic tissue by driving electrodes 24 to drive an electric current into the pelvic site of the subject having a frequency of between 3 Hz and 50 Hz, for example, between 10 Hz and 20 Hz, and a current of between 1 mA and 7 mA, for example, between 1 mA and 3 mA.

For some applications, device 20 is inserted by the subject in accordance with a schedule related to the timing of ovulation of the subject, e.g., within 3 days of the start or end of ovulation. In such an application, device 20 is inserted in order to enhance the fertility of the subject, e.g., by increasing blood circulation due to increasing nitric oxide. For some applications, sensor 23 is an ovulation sensor that senses ovulation of the subject and generates a signal in response thereto. The control unit receives the signal and increases the nitric oxide production in response to receiving the signal.

For some applications, insertion device 20 is inserted by a subject identified as having a multiple gestation pregnancy. In such an application, insertion device 20 is inserted in order to increase the blood supply to the fetuses, by enhancing nitric oxide production, even in the absence of detection of a decline in fetal heart rate. For some applications, insertion device 20 is inserted in order to increase the blood supply to a single fetus, even in the absence of detection of a decline in fetal heart rate. In another application, insertion device 20 is automatically or manually activated, or inserted by the subject, in response to a sensed decline in the heartbeat of a fetus. In one application, sensor 23 comprises an ultrasound sensor. In another application, the sensor comprises another suitable sensor for assessing fetal heartbeat. In such an application, insertion device 20 is activated or inserted in order to increase the blood supply to the fetus.

Reference is now made to FIG. 2, which is a schematic illustration of a nerve cuff 30 configured to be coupled to a nerve 32 innervating a uterus of the subject. For example, cuff 30 may be placed around the inferior hypogastric plexus, the uterovaginal plexus, and/or the pelvic splanchnic nerve of the subject. A set of one or more electrodes 24 is disposed on the surface of nerve cuff 30, in contact with or near nerve 32 following implantation. Nerve cuff 30 is typically configured to be surgically implanted on nerve 32 of the subject, using techniques that are known in the art.

For such applications, control unit 22 typically comprises a handheld wand that is activated by the subject in response to the onset of heavy bleeding and/or in response to pain associated with menorrhagia. Alternatively or additionally, nerve cuff 30 or another portion of the apparatus comprises sensor 23, e.g., a pH sensor, an electrical sensor, an electromyographic sensor that senses muscle activity associated with menstrual cramps, or a mechanical sensor, each of which is configured to alert the control unit of the initiation of heavy bleeding and/or of menstrual pain. In response to the signal from the wand, or from the sensor, control unit 22 is configured to treat the pain and/or bleeding by, for example, driving a current via electrodes 24 into nerve 32 of the subject, to reduce nitric oxide production by pelvic tissue of the subject.

Typically, control unit 22 drives electrodes 24 to drive into nerve 32 an electric current having a frequency of between 50 Hz and 150 Hz (for example, between 50 Hz and 100 Hz). Further typically, control unit 22 drives each of the electrodes 24 to drive a current into the nerve having an amplitude of between 1 mA and 6 mA, e.g., between 2 mA and 4 mA.

For some applications, control unit 24 is configured to drive a current for reducing pain associated with menorrhagia. In accordance with respective applications, the current may be driven via the set of one or more electrodes 24 coupled to nerve cuff 30, or via electrodes 24 of insertion device 20. The current is typically driven into a pelvic muscle of the subject (e.g., a pelvic floor muscle), and has different signal parameters from those used to treat menorrhagia. Further typically, control unit 24 is configured to drive a collision blocking current to reduce pain associated with menorrhagia.

For some applications, techniques described in the present patent application are practiced in combination with techniques described in one or more of the references cited in the Background section of the present patent application.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating menorrhagia of a subject, comprising:
at least one electrode, configured to be coupled to a pelvic site of the subject; and
a control unit, configured to reduce nitric oxide production by pelvic tissue of the subject by driving the electrode to drive an electric current into the pelvic site of the subject.

2. The apparatus according to claim 1, further comprising a sensor configured to sense a level of bleeding of the subject and to generate a signal in response thereto, wherein the control unit is configured to receive the signal and to reduce the nitric oxide production in response to the signal.

3. The apparatus according to claim 1, wherein the control unit is configured to configure the electric current to have a frequency that is between 50 Hz and 150 Hz.

4. The apparatus according to claim 1, wherein the at least one electrode is configured to be inserted by the subject into a vagina of the subject and removed by the subject from the vagina of the subject.

5. The apparatus according to claim 4, wherein the at least one electrode is configured to be inserted into the vagina of the subject in accordance with a schedule related to timing of a menstrual period of the subject.

6. The apparatus according to claim 4, wherein a total length of the apparatus is 4-10 cm.

7. The apparatus according to claim 4, wherein the control unit is configured to configure the electric current to have an amplitude that is between 1 mA and 15 mA.

8. The apparatus according to claim 7, wherein the control unit is configured to configure the electric current to have an amplitude that is between 5 mA and 15 mA.

9. The apparatus according to claim 1, wherein the at least one electrode is configured to be coupled to a nerve that innervates a uterus of the subject.

10. The apparatus according to claim 9, wherein the at least one electrode is configured to be coupled to a nerve of the subject selected from the group consisting of an inferior hypogastric plexus, a uterovaginal plexus, and a pelvic splanchnic nerve.

11. The apparatus according to claim 9, wherein the control unit is configured to drive a pain-reduction current into the nerve of the subject to reduce pain associated with menorrhagia.

12. The apparatus according to claim 11, wherein the control unit is configured to drive into the nerve of the subject a collision blocking current configured to reduce pain associated with the menorrhagia.

13. The apparatus according to claim 11, wherein the control unit is configured to reduce nitric oxide production by using a first signal protocol, and to reduce pain by driving the pain reduction current into the subject's nerve using a second signal protocol, different from the first signal protocol.

14. A method, comprising:
identifying a subject as suffering from menorrhagia; and
in response to the identifying:
driving an electric current into a pelvic site of the subject; and
configuring the electric current to reduce nitric oxide production by pelvic tissue of the subject.

15. The method according to claim 14, wherein configuring the electric current comprises sensing a level of bleeding of the subject and configuring the electric current in response to the sensed level of bleeding.

16. The method according to claim 14, wherein configuring the electric current comprises sensing a level of pain of the subject and configuring the electric current in response to the sensed level of pain.

17. The method according to claim 14, wherein driving the electric current into the pelvic site of the subject comprises configuring the electric current to have a frequency that is between 50 Hz and 150 Hz.

18. The method according to claim 14, wherein driving the electric current into the pelvic site of the subject comprises configuring the electric current to have an amplitude that is between 1 mA and 15 mA.

19. The method according to claim 18, wherein driving the electric current into the pelvic site of the subject comprises configuring the electric current to have an amplitude that is between 5 mA and 15 mA.

20. The method according to claim 14, wherein the pelvic site includes a vagina of the subject, wherein the method further comprises inserting an electrode into the vagina, and wherein driving the electric current comprises driving the electric current via the electrode while the electrode is inserted.

21. The method according to claim 20, wherein inserting the electrode comprises inserting the electrode into the vagina of the subject in accordance with a schedule related to timing of a menstrual period of the subject.

22. The method according to claim 14, further comprising implanting the electrode in the pelvic site of the subject, wherein driving the electric current comprises driving the electric current via the electrode while the electrode is implanted in the pelvic site.

23. The method according to claim 22, wherein implanting the electrode comprises coupling the electrode to a nerve that innervates a uterus of the subject, and wherein driving the electric current comprises driving the electric current into the nerve.

24. The method according to claim 23, wherein implanting the electrode comprises coupling the electrode to a nerve of the subject selected from the group consisting of an inferior hypogastric plexus, a uterovaginal plexus, and a pelvic splanchnic nerve.

25. The method according to claim 23, wherein driving the electric current into the subject's nerve comprises driving an electric current to reduce pain associated with menorrhagia.

26. The method according to claim 25, wherein driving the electric current into the subject's nerve comprises reducing pain associated with the menorrhagia by driving a collision blocking current.

27. The method according to claim 25, wherein driving the electric current into the subject's nerve comprises driving an electric current using a first signal protocol to reduce nitric oxide production, and driving the electric current to reduce pain associated with menorrhagia using a second signal protocol, different from the first signal protocol.

28. Apparatus for treating menstrual pain of a subject, comprising:
at least one menstrual cramp sensor, configured to be coupled to a pelvic site of the subject and to generate a signal indicative of menstrual pain; and
a control unit, configured to receive the signal and to relieve the menstrual pain by driving an electric current into tissue of the subject.

29. The apparatus according to claim 28, wherein the apparatus comprises a manual insertion device, which comprises the sensor and the control unit.

30. The apparatus according to claim 28, wherein the apparatus is configured to be surgically implanted in the subject.

31. The apparatus according to claim 28, wherein the sensor comprises an electrode.

32. The apparatus according to claim 31, wherein the control unit is configured to receive an electromyographic signal from the electrode, which varies in response to menstrual cramps of the subject.

33. The apparatus according to claim 31, wherein the control unit is configured to drive the electric current via the electrode.

34. The apparatus according to claim 33, wherein the control unit is configured to reduce nitric oxide production by pelvic tissue of the subject, by driving the electrode to drive the electric current.

35. Apparatus for increasing fertility of a subject, comprising:
at least one electrode, configured to be coupled to a pelvic site of the subject; and
a control unit, configured to increase fertility of the subject by increasing nitric oxide production by tissue of the subject by driving an electric current into the tissue.

36. The apparatus according to claim 35, further comprising an ovulation sensor, configured to sense ovulation of the subject and to generate a signal in response thereto, wherein the control unit is configured to receive the signal and to increase the nitric oxide production in response to receiving the signal.

37. The apparatus according to claim 35, wherein the at least one electrode is configured to be inserted by the subject into a vagina of the subject and removed by the subject from the vagina of the subject.

38. The apparatus according to claim 35, wherein the control unit is configured to configure the current to have a frequency that is between 3 Hz and 50 Hz.

39. The apparatus according to claim 38, wherein the control unit is configured to configure the current to have a frequency that is between 10 Hz and 20 Hz.

40. The apparatus according to claim 35, wherein the control unit is configured to configure the current to have an amplitude that is between 1 mA and 7 mA.

41. The apparatus according to claim 40, wherein the control unit is configured to configure the current to have an amplitude that is between 1 mA and 3 mA.

42. A method for increasing fertility of a subject, comprising:
identifying a subject who wants to become pregnant; and
in response to the identifying, driving an electric current into a pelvic site of the subject configured to increase fertility of the subject by enhancing nitric oxide production by pelvic tissue of the subject.

43. The method according to claim 42, further comprising sensing ovulation of the subject, and driving the current in response to sensing the ovulation.

44. The method according to claim 42, wherein the pelvic site includes a vagina of the subject, wherein the method further comprises inserting an electrode into the vagina, and wherein driving the electric current comprises driving the electric current via the electrode while the electrode is inserted.

45. The method according to claim 44, wherein inserting the electrode comprises inserting the electrode into the vagina of the subject in accordance with a schedule related to timing of ovulation of the subject.

46. The method according to claim 42, wherein driving the electric current comprises configuring the electric current to have a frequency that is between 3 Hz and 50 Hz.

47. The method according to claim 46, wherein driving the electric current comprises configuring the electric current to have a frequency that is between 10 Hz and 20 Hz.

48. The method according to claim 42, wherein driving the electric current comprises configuring the electric current to have an amplitude that is between 1 mA and 7 mA.

49. The method according to claim 48, wherein driving the electric current comprises configuring the electric current to have an amplitude that is between 1 mA and 3 mA.

50. Apparatus for increasing blood supply to a fetus within a subject, comprising:
at least one electrode configured to be coupled to a pelvic site of the subject; and
a control unit, configured to enhance fetal blood supply by increasing nitric oxide production of tissue of the subject, by driving the electrode to drive an electric current into the pelvic site of the subject.

51. The apparatus according to claim 50, further comprising a sensor configured to sense a heartbeat of the fetus and to generate a signal indicative of the fetal heartbeat, wherein the control unit is configured to enhance the fetal blood supply in response to identifying a decline of the fetal heartbeat.

52. The apparatus according to claim 50, wherein the apparatus is configured to be surgically implanted in the subject.

53. The apparatus according to claim 50, wherein the at least one electrode is configured to be inserted by the subject into a vagina of the subject and removed by the subject from the vagina of the subject.

54. The apparatus according to claim 50, wherein the control unit is configured to configure the current to have a frequency that is between 3 Hz and 50 Hz.

55. The apparatus according to claim 54, wherein the control unit is configured to configure the current to have a frequency that is between 10 Hz and 20 Hz.

56. The apparatus according to claim 50, wherein the control unit is configured to configure the current to have an amplitude that is between 1 mA and 7 mA.

57. The apparatus according to claim 56, wherein the control unit is configured to configure the current to have an amplitude that is between 1 mA and 3 mA.

58. A method, comprising:
identifying a subject who is pregnant with a fetus; and
in response to the identifying, driving an electric current into a pelvic site of the subject configured to increase blood supply to the fetus by enhancing nitric oxide production by pelvic tissue of the subject.

59. The method according to claim 58, further comprising surgically implanting an electrode in the subject, wherein driving the current comprises driving the current via the electrode.

60. The method according to claim 58, further comprising sensing a decline in fetal heartbeat, wherein driving the current comprises driving the current in response to sensing the decline.

61. The method according to claim 58, wherein the pelvic site includes a vagina of the subject, wherein the method further comprises inserting an electrode into the vagina, and wherein driving the electric current comprises driving the electric current via the electrode while the electrode is inserted.

62. The method according to claim 58, wherein driving the electric current comprises configuring the electric current to have a frequency that is between 3 Hz and 50 Hz.

63. The method according to claim 62, wherein driving the electric current comprises configuring the electric current to have a frequency that is between 10 Hz and 20 Hz.

64. The method according to claim 58, wherein driving the electric current comprises configuring the electric current to have a frequency that is between 1 mA and 7 mA.

65. The method according to claim 64, wherein driving the electric current comprises configuring the electric current to have a frequency that is between 1 mA and 3 mA.

66. The method according to claim 58, wherein driving the current comprises driving the current even in the absence of sensing a decline in fetal heartbeat within an immediately preceding 30 minute period.

67. The method according to claim 66, wherein identifying comprises identifying that the subject is pregnant with more than one fetus.
